SEQUENCE LISTING (28 sequences)

SEQ ID NO: 1 - protein, 5 aa, Artificial Sequence
  Description of Artificial Sequence Synthetic peptide
SYAMS

SEQ ID NO: 2 - protein, 16 aa, Artificial Sequence
  Description of Artificial Sequence Synthetic peptide
SISKGGNTYYADSVKG

SEQ ID NO: 3 - protein, 10 aa, Artificial Sequence
  Description of Artificial Sequence Synthetic peptide
GWGDYGWFAY

SEQ ID NO: 4 - protein, 7 aa, Artificial Sequence
  Description of Artificial Sequence Synthetic peptide
GFTFSSY

SEQ ID NO: 5 - protein, 5 aa, Artificial Sequence
  Description of Artificial Sequence Synthetic peptide
SKGGN

SEQ ID NO: 6 - protein, 10 aa, Artificial Sequence
  Description of Artificial Sequence Synthetic peptide
GWGDYGWFAY

SEQ ID NO: 7 - protein, 8 aa, Artificial Sequence
  Description of Artificial Sequence Synthetic peptide
GFTFSSYA

SEQ ID NO: 8 - protein, 7 aa, Artificial Sequence
  Description of Artificial Sequence Synthetic peptide
ISKGGNT

SEQ ID NO: 9 - protein, 13 aa, Artificial Sequence
  Description of Artificial Sequence Synthetic peptide
ARGWGDYGWFAYW

SEQ ID NO: 10 - protein, 10 aa, Artificial Sequence
  Description of Artificial Sequence Synthetic peptide
GFTFSSYAMS

SEQ ID NO: 11 - protein, 9 aa, Artificial Sequence
  Description of Artificial Sequence Synthetic peptide
SISKGGNTY

SEQ ID NO: 12 - protein, 10 aa, Artificial Sequence
  Description of Artificial Sequence Synthetic peptide
GWGDYGWFAY

SEQ ID NO: 13 - protein, 11 aa, Artificial Sequence
  Description of Artificial Sequence Synthetic peptide
KASQDINRYLN

SEQ ID NO: 14 - protein, 7 aa, Artificial Sequence
  Description of Artificial Sequence Synthetic peptide
RANRLLD

SEQ ID NO: 15 - protein, 9 aa, Artificial Sequence
  Description of Artificial Sequence Synthetic peptide
LQYDEFPLT

SEQ ID NO: 16 - protein, 11 aa, Artificial Sequence
  Description of Artificial Sequence Synthetic peptide
KASQDINRYLN

SEQ ID NO: 17 - protein, 7 aa, Artificial Sequence
  Description of Artificial Sequence Synthetic peptide
RANRLLD

SEQ ID NO: 18 - protein, 9 aa, Artificial Sequence
  Description of Artificial Sequence Synthetic peptide
LQYDEFPLT

SEQ ID NO: 19 - protein, 6 aa, Artificial Sequence
  Description of Artificial Sequence Synthetic peptide
QDINRY

SEQ ID NO: 20 - protein, 3 aa, Artificial Sequence
  Description of Artificial Sequence Synthetic peptide
RAN

SEQ ID NO: 21 - protein, 9 aa, Artificial Sequence
  Description of Artificial Sequence Synthetic peptide
LQYDEFPLT

SEQ ID NO: 22 - protein, 11 aa, Artificial Sequence
  Description of Artificial Sequence Synthetic peptide
KASQDINRYLN

SEQ ID NO: 23 - protein, 7 aa, Artificial Sequence
  Description of Artificial Sequence Synthetic peptide
RANRLLD

SEQ ID NO: 24 - protein, 9 aa, Artificial Sequence
  Description of Artificial Sequence Synthetic peptide
LQYDEFPLT

SEQ ID NO: 25 - protein, 118 aa, Artificial Sequence
  Description of Artificial Sequence Synthetic polypeptide
QVQLVESGGGVVQPGRSLRLSCAASGFTFSSYAMSWVRQAPGKGLEWVASISKGGNTYYADSVKGRFTISRDNSKNTLYLQMNSLRAEDTAVYYCARGWGDYGWFAYWGQVTLVTVSS

SEQ ID NO: 26 - protein, 107 aa, Artificial Sequence
  Description of Artificial Sequence Synthetic polypeptide
DIQMTQSPSSLSASVGDRVTITCKASQDINRYLNWFQQKPGKAPKSLIYRANRLLDGVPSRFSGSGSGTDFTLTISSLQPEDFATYYCLQYDEFPLTFGQGTKLEIK

SEQ ID NO: 27 - protein, 448 aa, Artificial Sequence
  Description of Artificial Sequence Synthetic polypeptide
QVQLVESGGGVVQPGRSLRLSCAASGFTFSSYAMSWVRQAPGKGLEWVASISKGGNTYYADSVKGRFTISRDNSKNTLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 214 aa, Artificial Sequence
  Description of Artificial Sequence Synthetic polypeptide
DIQMTQSPSSLSASVGDRVTITCKASQDINRYLNWFQQKPGKAPKSLIYRANRLLDGVPSRFSGSGSGTDFTLTISSLQPEDFATYYCLQYDEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0127346 A1
(43) Pub. Date: Apr. 28, 2022

(54) Methods of Safe Administration of Anti-Tau Antibody

(71) Applicant: JANSSEN PHARMACEUTICA NV, Beerse (BE)

(72) Inventors: David Benjamin Henley, Hagerstown, IN (US); Partha Nandy, Titusville, NJ (US); Carlos Pérez Ruixo, Valencia (ES)

(21) Appl. No.: 17/509,910

(22) Filed: Oct. 25, 2021

Related U.S. Application Data
(60) Provisional application No. 63/105,810, filed on Oct. 26, 2020.
(60) Provisional application No. 63/250,114, filed on Sep. 29, 2021.

(51) Int. Cl. C07K 16/18; A61P 25/28

(52) U.S. Cl. CPC C07K 16/18; C07K 2317/565; A61P 25/28

(57) ABSTRACT

Methods are described for safely administering to a subject in need thereof an anti-tau antibody that bind to tau, in particular that bind to a phosphorylated epitope on tau. The methods comprise administering a pharmaceutical composition comprising the anti-tau antibody, in which the anti-tau antibody is administered in an amount of about 500 mg to 5000 mg per dose.

### CROSS-REFERENCE TO RELATED APPLICATIONS

This application claims the benefit of U.S. Provisional Application No. 63/105,810, filed on Oct. 26, 2020, and of U.S. Provisional Application No. 63/250,114, filed on Sep. 29, 2021, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Oct. 6, 2021, is named JNJ_010_US1-SL.txt and is 13,824 bytes in size.

### FIELD OF THE INVENTION

The present invention is in the field of medical treatment. In particular, the invention relates to anti-tau antibodies and their administration to human subjects.

### BACKGROUND

Alzheimer's Disease is a neurodegenerative disease characterized by cognitive deficits and memory loss, as well as behavioral and psychiatric symptoms that include anxiety, depression, and agitation. This disease is associated with aging and is believed to represent the fourth most common medical cause of death in the United States.

The hallmark pathological features of Alzheimer's Disease are amyloid plaques and neurofibrillary tangles. Amyloid plaques primarily consist of beta-amyloid (Aβ). Many therapies currently in development aimed at modifying or slowing the progression of Alzheimer's Disease are targeting Aβ. Such therapies include Eli Lilly's solanezumab, Biogen's aducanumab, and Roche's crenezumab, which are all humanized monoclonal antibodies against amyloid beta (Aβ).

Neurofibrillary tangles consist of aggregates of hyperphosphorylated tau protein and are generally found in several areas of the human brain of patients with Alzheimer's Disease that are important for memory and cognitive function. The main physiological function of tau is microtubule polymerization and stabilization. The binding of tau to microtubules occurs by ionic interactions between positive charges in the microtubule binding region of tau and negative charges on the microtubule lattice (Butner and Kirschner 1991). Tau protein contains 85 possible phosphorylation sites and phosphorylation at many of these sites interferes with the primary function of tau. Tau that is bound to the axonal microtubule lattice is in a hypo-phosphorylation state, while aggregated tau in Alzheimer's Disease is hyper-phosphorylated.

Several candidate drugs that prevent or clear tau aggregation are currently in development (Brunden et al. 2009). Studies in transgenic mice models have shown that both active and passive tau immunization can have beneficial therapeutic effects (Asuni et al. 2007; Boutajangout et al. 2011). Further, activity has been reported with both phospho-directed and non-phospho-directed antibodies (Schroeder et al. 2016).

However, studies on the safety of tau immunotherapies are still ongoing and a mechanistic understanding of the efficacy and safety of the various approaches is not well established (Sigurdsson 2016). Thus, there remains a need for safe therapeutics that prevent tau aggregation and tauopathy progression to treat tauopathies such as Alzheimer's Disease.

### SUMMARY OF THE INVENTION

Some of the main aspects of the present invention are summarized below. Additional aspects are described in the Detailed Description of the Invention, Example, and Claims sections of this disclosure. The description in each section of this disclosure is intended to be read in conjunction with the other sections. Furthermore, the various embodiments described in each section of this disclosure can be combined in various ways, and all such combinations are intended to fall within the scope of the present invention.

Accordingly, the disclosure provides methods of administering a monoclonal antibody that binds to tau, preferably phosphorylated tau, to a subject.

One aspect of the invention relates to a method of administering a monoclonal antibody to a subject in need thereof, the method comprising administering to the subject a pharmaceutical composition comprising the monoclonal antibody and a pharmaceutically acceptable carrier, in which the monoclonal antibody is administered in an amount of about 500 mg to about 5000 mg per dose.

Another aspect of the invention relates to a pharmaceutical composition comprising a monoclonal antibody and a pharmaceutically acceptable carrier for use in administering the monoclonal antibody to a subject in need thereof, in which the monoclonal antibody is administered in an amount of about 500 mg to about 5000 mg per dose.

The monoclonal antibody for use in the methods of the present invention and the pharmaceutical compositions of the present invention may comprise: a heavy chain variable complementarity-determining region (CDR) 1 comprising the amino acid sequence of SEQ ID NO: 1, a heavy chain variable CDR2 comprising the amino acid sequence of SEQ ID NO: 2, a heavy chain variable CDR3 comprising the amino acid sequence of SEQ ID NO: 3, a light chain variable CDR1 comprising the amino acid sequence of SEQ ID NO: 13, a light chain variable CDR2 comprising the amino acid sequence of SEQ ID NO: 14, and a light chain variable CDR3 comprising the amino acid sequence of SEQ ID NO: 15. In some embodiments, the monoclonal antibody comprises a heavy chain variable CDR1 having the amino acid sequence of SEQ ID NO: 1, a heavy chain variable CDR2 having the amino acid sequence of SEQ ID NO: 2, a heavy chain variable CDR3 having the amino acid sequence of SEQ ID NO: 3, a light chain variable CDR1 having the amino acid sequence of SEQ ID NO: 13, a light chain variable CDR2 having the amino acid sequence of SEQ ID NO: 14, and a light chain variable CDR3 having the amino acid sequence of SEQ ID NO: 15.

The monoclonal antibody may comprise a heavy chain variable region comprising the amino acid sequence of SEQ ID NO: 25, and a light chain variable region comprising the amino acid sequence of SEQ ID NO: 26. In certain embodiments, the monoclonal antibody comprises a heavy chain variable region having the amino acid sequence of SEQ ID NO: 25, and a light chain variable region having the amino acid sequence of SEQ ID NO: 26.

Further, the monoclonal antibody may comprise a heavy chain comprising the amino acid sequence of SEQ ID NO: 27, and a light chain comprising the amino acid sequence of SEQ ID NO: 28. In certain embodiments, the monoclonal antibody comprises a heavy chain having the amino acid sequence of SEQ ID NO: 27, and a light chain having the amino acid sequence of SEQ ID NO: 28.

In addition to the monoclonal antibody, the composition may contain histidine, sucrose, polysorbate 20, and ethylenediamine tetra-acetic acid. The composition may have a pH of about 5-6.

In the methods or pharmaceutical compositions of the invention, the monoclonal antibody may be administered in an amount of about 1000 mg to about 3000 mg, or about 2000 mg to about 5000 mg, or about 3000 mg to about 5000 mg, per dose. In certain embodiments, the monoclonal antibody may be administered in an amount of about 500 mg, 750 mg, 1000 mg, 1200 mg, 1250 mg, 1400 mg, 1500 mg, 1600 mg, 1750 mg, 1800 mg, 2000 mg, 2200 mg, 2250 mg, 2400 mg, 2500 mg, 2600 mg, 2750 mg, 2800 mg, 3000 mg, 3200 mg, 3250 mg, 3400 mg, 3500 mg, 3600 mg, 3750 mg, 3800 mg, 4000 mg, 4200 mg, 4250 mg, 4400 mg, 4500 mg, 4600 mg, 4750 mg, 4800 mg, or 5000 mg, or any value in between, per dose.

The composition may be administered subcutaneously or by intravenous infusion. Further, the composition may be administered as more than one dose, for example, as more than one dose in which each dose is separated by a period of about 4 weeks.

In the methods or pharmaceutical compositions of the present invention, the subject may be in need of a treatment of Alzheimer's Disease. In particular embodiments, the subject may be in need of a treatment of early Alzheimer's Disease, prodromal Alzheimer's Disease, or mild Alzheimer's Disease.

### BRIEF DESCRIPTION OF THE FIGURES

FIG. 1 shows a schematic overview of the design of the study in Example 3.

### DETAILED DESCRIPTION OF THE INVENTION

The practice of the present invention will employ, unless otherwise indicated, conventional techniques of immunology, pharmaceutics, formulation science, cell biology, molecular biology, clinical pharmacology, and clinical practice, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Systeme International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10. Where a numeric term is preceded by “about,” the term includes the stated number and values ±10% of the stated number.

As used herein, the term “antibody” or “immunoglobulin” is used in a broad sense and includes immunoglobulin or antibody molecules including polyclonal antibodies, monoclonal antibodies including murine, human, human-adapted, humanized, and chimeric monoclonal antibodies and antibody fragments. In general, antibodies are proteins or peptide chains that exhibit binding specificity to a specific antigen. Antibody structures are well known. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species can be assigned to one of two clearly distinct types, namely kappa and lambda, based on the amino acid sequences of their constant domains.

In addition to the heavy and light constant domains, antibodies contain light and heavy chain variable regions. An immunoglobulin light or heavy chain variable region consists of a “framework” region interrupted by “antigen-binding sites.” The antigen-binding sites are defined using various terms and numbering schemes as follows:
(i) Kabat numbering scheme: “Complementarity Determining Regions” or “CDRs” are based on sequence variability (Wu and Kabat 1970). Generally, the antigen-binding site has three CDRs in each variable region (e.g., HCDR1, HCDR2 and HCDR3 in the heavy chain variable region (VH) and LCDR1, LCDR2 and LCDR3 in the light chain variable region (VL)).
(ii) Chothia numbering scheme: The term “hypervariable region,” “HVR” or “HV” refers to the regions of an antibody variable domain which are hypervariable in structure as defined by Chothia and Lesk (Chothia and Lesk 1987). Generally, the antigen-binding site has three hypervariable regions in each VH (H1, H2, H3) and VL (L1, L2, L3). Numbering systems as well as annotation of CDRs and HVs have been revised by Abhinandan and Martin (Abhinandan and Martin 2008).
(iii) IMGT numbering scheme: Proposed by Lefranc (Lefranc et al. 2003), regions that form the antigen-binding site are defined based on the comparison of V domains from immunoglobulins and T-cell receptors. The International ImMunoGeneTics (IMGT) database provides a standardized numbering and definition of these regions. The correspondence between CDRs, HVs and IMGT delineations is described in Lefranc et al.
(iv) Martin numbering scheme (also known as ABM numbering scheme): A compromise between Kabat and Chothia numbering schemes as described by Martin (Martin 2010).
(v) The antigen-binding site can be delineated based on “Specificity Determining Residue Usage” (SDRU) (Almagro 2004), where SDR, refers to amino acid residues of an immunoglobulin that are directly involved in antigen contact.

The term “pharmaceutical composition” refers to a preparation that is in such form as to permit the biological activity of the active ingredient to be effective and which contains no additional components that are unacceptably toxic to a subject to which the composition would be administered. Such composition can be sterile and can comprise a pharmaceutically acceptable carrier, such as physiological saline. In some embodiments, a pharmaceutically acceptable carrier can comprise a mixture, for example, a mixture of saline and buffer solution, etc. Suitable pharmaceutical compositions can comprise one or more of a buffer (e.g., acetate, phosphate or citrate buffer), a surfactant (e.g., polysorbate), a stabilizing agent (e.g., polyol or amino acid), a preservative (e.g., sodium benzoate), and/or other conventional solubilizing or dispersing agents.

As used herein, the term “tau” or “tau protein”, also known as microtubule-associated protein tau, MAPT, neurofibrillary tangle protein, paired helical filament (PHF)-tau, MAPTL, or MTBT1, refers to an abundant central and peripheral nervous system protein having multiple isoforms. In the human central nervous system (CNS), six major tau isoforms ranging in size from 352 to 441 amino acids in length exist due to alternative splicing (Hanger et al. 2009). Examples of tau include, but are not limited to, tau isoforms in the CNS, such as the 441-amino acid longest tau isoform (4R2N), also named microtubule-associated protein tau isoform 2, that has four repeats and two inserts, such as the human tau isoform 2 having the amino acid sequence represented in GenBank Accession No. NP 005901.2. Other examples of tau include the 352-amino acid long shortest (fetal) isoform (3R0N), also named microtubule-associated protein tau isoform 4, that has three repeats and no inserts, such as the human tau isoform 4 having the amino acid sequence represented in GenBank Accession No. NP_058525.1. Examples of tau also include the “big tau” isoform expressed in peripheral nerves that contains 300 additional residues (exon 4a) (Friedhoff et al. 2000). Examples of tau include a human big tau that is a 758 amino acid-long protein encoded by an mRNA transcript 6762 nucleotides long (NM_016835.4), or isoforms thereof. The amino acid sequence of the exemplified human big tau is represented in GenBank Accession No. NP_058519.3. As used herein, the term “tau” includes homologs of tau from species other than human, such as Macaca Fascicularis (cynomolgus monkey), rhesus monkeys or Pan troglodytes (chimpanzee). As used herein, the term “tau” includes proteins comprising mutations, e.g., point mutations, fragments, insertions, deletions, and splice variants of full-length wild type tau. The term “tau” also encompasses post-translational modifications of the tau amino acid sequence. Post-translational modifications include, but are not limited to, phosphorylation.

As used herein, the term “phosphorylated tau” refers to tau that has been phosphorylated on an amino acid residue at one or more locations of the amino acid sequence of tau. The phosphorylated amino acid residues can be, for example, serine (Ser), threonine (Thr) or tyrosine (Tyr). The site on tau that is phosphorylated is preferably a site that is specifically phosphorylated in neurodegenerative diseases such as Alzheimer's Disease. Examples of sites of phosphorylated tau to which the anti-phosphorylated tau antibody binds include, for example, Tyr18, Thr181, Ser199, Ser202, Thr205, Thr212, Ser214, Thr217, Ser396, Ser404, Ser409, Ser422, Thr427. As used throughout the present application, the amino acid positions are given in reference to the sequence of human microtubule-associated protein tau isoform 2 having the amino acid sequence represented in GenBank Accession No. NP_005901.2. Abnormal phosphorylated tau aggregates readily into insoluble oligomers which are neurotoxic and contribute to neurodegeneration (Goedert et al. 1991). The oligomers progress to tangles of so-called paired helical filaments (PHF) (Alonso et al. 2001). The degree of neurofibrillary tangle pathology has been consistently shown to be correlated to the degree of dementia in AD subjects (Bierer et al. 1995; Braak and Braak 1991; Delacourte 2001).

As used herein, the terms “p181tau”, “p181+tau”, and “p-tau181” are used interchangeably and refer to tau that is phosphorylated at Thr181. Similarly, the terms “p217tau”, “p217+tau”, and “p-tau217” are used interchangeably and refer to tau that is phosphorylated at Thr217. The same nomenclature format can be used to refer to tau that is phosphorylated at different amino acid residues.

A “subject” or “individual” or “patient” is any subject, particularly a mammalian subject, for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc.

An “effective amount” of a therapy is an amount sufficient to carry out a specifically stated purpose, such as to elicit a desired biological or medicinal response in a subject.

The terms “reduce,” “inhibit,” “block,” and “suppress” are used interchangeably and refer to any statistically significant decrease in occurrence or activity or extent or volume, including full blocking or complete elimination of the occurrence or activity or extent or volume. For example, “inhibition” can refer to a decrease of about 10%, 20%, 30%, 40%, 50%, 60%, 70%, 80%, 90% or 100% in activity or occurrence. As another example, “reduction” can refer to a decrease of about 10%, 20%, 30%, 40%, 50%, 60%, 70%, 80%, 90% or 100% in extent or volume.

An “adverse event” (AE) is any untoward medical occurrence in a subject administered a medicinal (investigational or non-investigational) product. An AE does not necessarily have a causal relationship with the intervention. An AE can therefore be any unfavorable and unintended sign (including an abnormal finding), symptom, or disease temporally associated with the use of a medicinal (investigational or non-investigational) product, whether or not related to that medicinal (investigational or non-investigational) product. This includes any occurrence that is new in onset or aggravated in severity or frequency from the baseline condition, or abnormal results of diagnostic procedures, including laboratory test abnormalities. According to some embodiments of the invention, AEs can be categorized based on severity using the following definitions: mild (grade 1), referring to an AE in which there is an awareness of symptoms that are easily tolerated, causing minimal discomfort and not interfering with everyday activities; moderate (grade 2), referring to an AE in which there is sufficient discomfort present that causes interference with normal activity; and severe (grade 3), referring to an AE in which there is extreme distress, causing significant impairment of functioning or incapacitation and prevention of normal everyday activities.

A “serious adverse event” (SAE) is any untoward medical occurrence that at any dose:
results in death;
is life-threatening (the subject is at risk of death at the time of the event);
requires inpatient hospitalization or prolongation of existing hospitalization;
results in persistent or significant disability/incapacity;
is a congenital anomaly/birth defect;
is a suspected transmission of any infectious agent via a medicinal product; or
is medically important (based on an exercise of medical and scientific judgment, such as an important medical event that may not be immediately life-threatening or result in death or hospitalization but may jeopardize the subject or may require intervention to prevent one of the other outcomes listed above).

### Anti-Tau Antibodies

The present invention relates to the administration of a monoclonal antibody that binds to tau. The anti-tau antibody can bind to a phosphorylated epitope on tau or bind to a non-phosphorylated epitope on tau.

In some embodiments, the anti-tau antibody can bind to a phosphorylated tau protein at an epitope in the proline rich domain of the tau protein. In certain embodiments, the anti-tau antibody can bind to a phosphorylated tau protein at an epitope comprising phosphorylated Thr181, Thr212, and/or Thr217 residues.

In embodiments of the invention, the anti-tau antibody may comprise heavy chain variable CDRs and light chain variable CDRs as shown in Table 1 below.

TABLE 1
Sequences for the heavy chain variable CDRs and light chain
variable CDRs of the anti-tau antibody.

| | | | |
|---|---|---|---|
| Variable Region | CDR1 | CDR2 | CDR3 |

Kabat numbering scheme

| | | | |
|---|---|---|---|
| Heavy Chain | SYAMS | SISKGGNTYYADSVKG | GWGDYGWFAY |
| | (SEQ ID NO: 1) | (SEQ ID NO: 2) | (SEQ ID NO: 3) |
| Light Chain | KASQDINRYLN | RANRLLD | LQYDEFPLT |
| | (SEQ ID NO: 13) | (SEQ ID NO: 14) | (SEQ ID NO: 15) |

Chothia numbering scheme

| | | | |
|---|---|---|---|
| Heavy Chain | GFTFSSY | SKGGN | GWGDYGWFAY |
| | (SEQ ID NO: 4) | (SEQ ID NO: 5) | (SEQ ID NO: 6) |
| Light Chain | KASQDINRYLN | RANRLLD | LQYDEFPLT |
| | (SEQ ID NO: 16) | (SEQ ID NO: 17) | (SEQ ID NO: 18) |

IMGT numbering scheme

| | | | |
|---|---|---|---|
| Heavy Chain | GFTFSSYA | ISKGGNT | ARGWGDYGWFAYW |
| | (SEQ ID NO: 7) | (SEQ ID NO: 8) | (SEQ ID NO: 9) |
| Light Chain | QDINRY | RAN | LQYDEFPLT |
| | (SEQ ID NO: 19) | (SEQ ID NO: 20) | (SEQ ID NO: 21) |

ABM numbering scheme

| | | | |
|---|---|---|---|
| Heavy Chain | GFTFSSYAMS | SISKGGNTY | GWGDYGWFAY |
| | (SEQ ID NO: 10) | (SEQ ID NO: 11) | (SEQ ID NO: 12) |
| Light Chain | KASQDINRYLN | RANRLLD | LQYDEFPLT |
| | (SEQ ID NO: 22) | (SEQ ID NO: 23) | (SEQ ID NO: 24) |

Thus, according to embodiments of the invention, the anti-tau antibody comprises:
(a) a heavy chain variable CDR1 comprising the amino acid sequence of SEQ ID NOS: 1, 4, 7, or 10;
(b) a heavy chain variable CDR2 comprising the amino acid sequence of SEQ ID NOS: 2, 5, 8, or 11;
(c) a heavy chain variable CDR3 comprising the amino acid sequence of SEQ ID NOS: 3, 6, 9, or 12;
(d) a light chain variable CDR1 comprising the amino acid sequence of SEQ ID NOS: 13, 16, 19, or 22;
(e) a light chain variable CDR2 comprising the amino acid sequence of SEQ ID NOS: 14, 17, 20, or 23; and
(f) a light chain variable CDR3 comprising the amino acid sequence of SEQ ID NOS: 15, 18, 21, or 24.

In some embodiments, the anti-tau antibody comprises:
(a) a heavy chain variable CDR1 having the amino acid sequence of SEQ ID NOS: 1, 4, 7, or 10;
(b) a heavy chain variable CDR2 having the amino acid sequence of SEQ ID NOS: 2, 5, 8, or 11;
(c) a heavy chain variable CDR3 having the amino acid sequence of SEQ ID NOS: 3, 6, 9, or 12;
(d) a light chain variable CDR1 having the amino acid sequence of SEQ ID NOS: 13, 16, 19, or 22
(e) a light chain variable CDR2 having the amino acid sequence of SEQ ID NOS: 14, 17, 20, or 23; and
(f) a light chain variable CDR3 having the amino acid sequence of SEQ ID NOS: 15, 18, 21, or 24.

In certain embodiments, the anti-tau antibody comprises a heavy chain variable CDR1 comprising the amino acid sequence of SEQ ID NO: 1, a heavy chain variable CDR2 comprising the amino acid sequence of SEQ ID NO: 2, a heavy chain variable CDR3 comprising the amino acid sequence of SEQ ID NO: 3, a light chain variable CDR1 comprising the amino acid sequence of SEQ ID NO: 13, a light chain variable CDR2 comprising the amino acid sequence of SEQ ID NO: 14, and a light chain variable CDR3 comprising the amino acid sequence of SEQ ID NO: 15. In particular embodiments, the anti-tau antibody comprises a heavy chain variable CDR1 having the amino acid sequence of SEQ ID NO: 1, a heavy chain variable CDR2 having the amino acid sequence of SEQ ID NO: 2, a heavy chain variable CDR3 having the amino acid sequence of SEQ ID NO: 3, a light chain variable CDR1 having the amino acid sequence of SEQ ID NO: 13, a light chain variable CDR2 having the amino acid sequence of SEQ ID NO: 14, and a light chain variable CDR3 having the amino acid sequence of SEQ ID NO: 15.

In embodiments of the invention, the anti-tau antibody comprises a heavy chain variable comprising the amino acid sequence of SEQ ID NO: 25, and a light chain variable comprising the amino acid sequence of SEQ ID NO: 26. In certain embodiments, the anti-tau antibody comprises a heavy chain variable having the amino acid sequence of SEQ ID NO: 25, and a light chain variable comprising the amino acid sequence of SEQ ID NO: 26.

In embodiments of the invention, the anti-tau antibody is an immunoglobulin G (IgG) antibody. In certain embodiments, the anti-tau antibody is an IgG1 antibody. Alternatively, the anti-tau antibody is an IgG2, IgG3, or IgG4 antibody. In other embodiments, the anti-tau antibody is an IgA, IgD, IgE, or IgM antibody.

In embodiments of the invention, the anti-tau antibody comprises a kappa light chain constant region. In other embodiments, the anti-tau antibody comprises a delta light chain constant region.

In preferred embodiments, the anti-tau antibody is an IgG1 antibody having a kappa light chain constant region.

In embodiments of the invention, the anti-tau antibody comprises a heavy chain comprising the amino acid sequence of SEQ ID NO: 27, and a light chain comprising the amino acid sequence of SEQ ID NO: 28. In certain embodiments, the anti-tau antibody comprises a heavy chain having the amino acid sequence of SEQ ID NO: 27, and a light chain having the amino acid sequence of SEQ ID NO: 28.

In preferred embodiments, the anti-tau antibody is a humanized monoclonal antibody.

Anti-tau antibody of the present invention can be produced by a variety of techniques, for example by the hybridoma method (Köhler and Milstein 1975). Chimeric monoclonal antibodies containing a light chain and heavy chain variable region derived from a donor antibody (typically murine) in association with light and heavy chain constant regions derived from an acceptor antibody (typically another mammalian species such as human) can be prepared by a method disclosed in U.S. Pat. No. 4,816,567. CDR-grafted monoclonal antibodies having CDRs derived from a non-human donor immunoglobulin (typically murine) and the remaining immunoglobulin-derived parts of the molecule being derived from one or more human immunoglobulins can be prepared by techniques known to those skilled in the art such as that disclosed in U.S. Pat. No. 5,225,539. Fully human monoclonal antibodies lacking any non-human sequences can be prepared from human immunoglobulin transgenic mice by techniques referenced in (Lonberg et al. 1994; Fishwild et al. 1996; Mendez et al. 1997). Human monoclonal antibodies can also be prepared and optimized from phage display libraries (Knappik et al. 2000; Krebs et al. 2001; Shi et al. 2010).

In embodiments of the invention, the anti-tau antibody may be formulated in a composition comprising a pharmaceutically acceptable carrier. The composition may also comprise one or more pharmaceutically acceptable excipients, which are well known in the art (see Remington's Pharmaceutical Science 1980). The preferred formulation of the pharmaceutical composition depends on the intended mode of administration and therapeutic application. The pharmaceutically-acceptable carriers can be vehicles commonly used to formulate pharmaceutical compositions for animal or human administration. In addition, the pharmaceutical composition may also include other diluents, adjuvants, or nontoxic, nontherapeutic, non-immunogenic stabilizers, and the like. It will be understood that the characteristics of the carrier, excipient or diluent will depend on the route of administration for a particular application.

In certain embodiments, the composition may comprise one or more stabilizing agents (for example, dextran 40, sucrose, glycine, lactose, mannitol, trehalose, maltose), one or more buffers (for example, acetate, citrate, histidine, lactate, phosphate, Tris), one or more surfactants (for example, polysorbate, sodium lauryl sulfate, polyethylene glycol-fatty acid esters, lecithins), one or more chelators (for example, ethylenediamine tetra-acetic acid (EDTA), edetate sodium), and a carrier (for example, water for injection water, physiological phosphate-buffered saline, Ringer's solutions, dextrose solution, Hank's solution). In preferred embodiments, the composition comprises water for injection, histidine, sucrose, polysorbate 20, and EDTA. The composition may have a pH of about 4 to about 7, or about 5 to about 6, preferably about 5.5.

### Methods of Use

A general aspect of the present invention relates to methods of administering to the subject a composition comprising an anti-tau antibody according to embodiments of the invention. These methods may provide delivery of the anti-tau antibody to the subject in an effective and safe amount.

According to embodiments of the invention, the composition may be administered in an amount of about 50 mg to about 5000 mg per dose of the anti-tau antibody. In some embodiments, the composition may be administered in an amount of about 500 mg to about 5000 mg per dose, or about 1000 mg to about 3000 mg per dose, or about 2000 mg to about 5000 mg per dose, or about 3000 mg to about 5000 mg per dose, of the anti-tau antibody. In certain embodiments, the composition may be administered in an amount of about 50 mg, 100 mg, 250 mg, 500 mg, 750 mg, 1000 mg, 1200 mg, 1250 mg, 1400 mg, 1500 mg, 1600 mg, 1750 mg, 1800 mg, 2000 mg, 2200 mg, 2250 mg, 2400 mg, 2500 mg, 2600 mg, 2750 mg, 2800 mg, 3000 mg, 3200 mg, 3250 mg, 3400 mg, 3500 mg, 3600 mg, 3750 mg, 3800 mg, 4000 mg, 4200 mg, 4250 mg, 4400 mg, 4500 mg, 4600 mg, 4750 mg, 4800 mg, or 5000 mg, or any value in between, per dose of the anti-tau antibody.

In embodiments of the invention, the composition may be administered in an amount of about 1 mg/kg to about 60 mg/kg per dose of the anti-tau antibody. In some embodiments, the composition may be administered in an amount of about 10 mg/kg to about 40 mg/kg per dose, or about 20 mg/kg to about 60 mg/kg per dose, or about 40 mg/kg to about 60 mg/kg per dose, of the anti-tau antibody. In certain embodiments, the composition may be administered in an amount of about 1 mg/kg, 3 mg/kg, 5 mg/kg, 10 mg/kg, 12.5 mg/kg, 15 mg/kg, 20 mg/kg, 25 mg/kg, 30 mg/kg, 35 mg/kg, 37.5 mg/kg, 40 mg/kg, 45 mg/kg, 50 mg/kg, 55 mg/kg, 60 mg/kg, or any value in between, per dose of the anti-tau antibody.

According to some embodiments, the composition may be administered as more than one dose. In certain embodiments, administration of each dose may be separated by a period of time, for example, about 4 weeks.

The composition comprising the anti-tau antibody can be administered by parenteral, topical, oral, intra-arterial, intracranial, intraperitoneal, intradermal, intranasal, or intramuscular means for prophylactic and/or therapeutic treatment. In certain embodiments, the composition can be administered subcutaneously. In certain embodiments, the composition can be administered by intravenous infusion.

According to some embodiments, the subject is a human subject. In certain embodiments, the subject is a human subject in need of treatment of a neurodegenerative disease, disorder, or condition.

As used herein a “neurodegenerative disease, disorder, or condition” includes any neurodegenerative disease, disorder, or condition known to those skilled in the art in view of the present disclosure. Examples of neurodegenerative diseases, disorders, or conditions include neurodegenerative diseases or disorders caused by or associated with the formation of neurofibrillary lesions, such as tau-associated diseases, disorders or conditions, referred to as tauopathies. According to particular embodiments, the neurodegenerative disease, disorder, or condition includes any of the diseases or disorders that show co-existence of tau and/or amyloid pathologies including, but not is limited to, Alzheimer's Disease, Parkinson's Disease, Creutzfeldt-Jacob disease, Dementia pugilistica, Down(' s) Syndrome, Gerstmann-Straussler-Scheinker disease, inclusion body myositis, prion protein cerebral amyloid angiopathy, traumatic brain injury, amyotrophic lateral sclerosis, parkinsonism-dementia complex of Guam, Non-Guamanian motor neuron disease with neurofibrillary tangles, argyrophilic grain dementia, corticobasal degeneration, Dementia in Amyotrophic Lateral Sclerosis, diffuse neurofibrillary tangles with calcification, frontotemporal dementia, preferably frontotemporal dementia with parkinsonism linked to chromosome 17 (FTDP-17), frontotemporal lobar dementia, Hallevorden-Spatz disease, multiple system atrophy, Niemann-Pick disease type C, Pick's disease, progressive subcortical gliosis, progressive supranuclear palsy, Subacute sclerosing panencephalitis, Tangle only dementia, Postencephalitic Parkinsonism, Myotonic dystrophy, chronic traumatic encephalopathy (CTE), Primary age-related Tauopathy (PART), cerebral angiopathy or Lewy body dementia (LBD). According to particular embodiments, the neurodegenerative disease, disorder, or condition is Alzheimer's disease or another tauopathy. According to preferred embodiments, the neurodegenerative disease, disorder, or condition is Alzheimer' s Disease.

The clinical course of Alzheimer's Disease can be divided into stages, with progressive patterns of cognitive and functional impairments. The stages can be defined using grading scales known in the art including, for instance, NIA-AA Research Framework (see, e.g., Dubois et al. 2016; Dubois et al. 2014; Jack et al. 2018) and the Clinical Dementia Rating (CDR) scale (see, e.g., Berg 1988), the contents of each of which are hereby incorporated by reference in their entirety.

For example, National Institute on Aging-Alzheimer' s Association (NIA-AA) research framework defines Alzheimer's Disease biologically, by neuropathologic change or biomarkers, and treats cognitive impairment as a symptom/sign of the disease rather than the definition of the disease (see, e.g., Jack et al. 2018, the content of which is incorporated herein by reference). According to the NIA-AA definition, an individual with biomarker evidence of Aβ deposition alone (abnormal amyloid positron emission tomography (PET) scan or low cerebrospinal fluid (CSF) Aβ42 or Aβ42/Aβ40 ratio) with a normal pathologic tau biomarker would be assigned the label “Alzheimer's pathologic change,” and the term “Alzheimer's Disease” would be applied if both biomarker evidence of AP and pathologic tau are present. The NIA-AA also developed a system for staging severity of Alzheimer's Disease. In particular, under the NIA-AA definition (reproduced from Text Box 2 of Jack et al. 2018, supra):

Definition:
A: Aβ biomarkers determine whether or not an individual is in the Alzheimer's continuum.
T: Pathologic tau biomarkers determine if someone who is in the Alzheimer's continuum has Alzheimer's disease

Staging severity:
(N): Neurodegenerative/neuronal injury biomarkers
(C): Cognitive symptoms
   A and T indicate specific neuropathologic changes that define Alzheimer's disease, whereas (N) and (C) are not specific to Alzheimer's disease and are therefore placed in parentheses.

According to preferred embodiments, the neurodegenerative disease, disorder, or condition is early Alzheimer's Disease, prodromal Alzheimer's Disease (Alzheimer's Disease with mild cognitive impairment (MCI)), or mild Alzheimer's Disease (also referred to as mild Alzheimer's Disease dementia).

In some embodiments, the neurodegenerative disease, disorder, or condition is mild to moderate Alzheimer's Disease.

In some embodiments, the subject in need of a treatment is amyloid positive in the brain but does not yet show significant cognitive impairment. The amyloid deposition in the brain can be detected using methods known in the art, such as PET scan, immunoprecipitation mass spectrometry, or other methods (for example, use of CSF biomarkers) (Jack et al. 2018).

In other embodiments, the human subject in need of a treatment has abnormal level of CSF Aβ amyloid 42 (Aβ42) consistent with Alzheimer's Disease pathology. For example, the subject can have low level of CSF Aβ42 or low Aβ42/Aβ40 ratio consistent with Alzheimer's Disease pathology (see, e.g., Jack et al. 2018, supra).

In certain embodiments, the subject experienced a gradual and progressive subjective decline in cognition over at least the previous 6 months, was evaluated to have a CDR-Global Score (CDR-GS) of 0.5 and a memory box score ≥0.5. In some embodiments, the subject exhibits pathologically elevated plasma tau (T+). In certain embodiments, the subject exhibits evidence of pathologic tau on a screening tau PET scan.

In some embodiments, the human subject is in need of treatment of prodromal or mild Alzheimer's Disease. In certain embodiments, the subjects was evaluated to have a CDR-GS of 0.5 or 1.0. In certain embodiments, the subject showed evidence of amyloid deposition and/or tauopathy (as demonstrated by abnormal CSF Aβ1-42 and elevated CSF p-tau181 or total tau).

In some embodiments, the pharmaceutical composition may be administered without inducing a serious adverse event in the subject. In certain embodiments, the pharmaceutical composition may be administered without inducing a severe adverse event in the subject.

In some embodiments, the anti-tau antibody is administered in an effective amount to reduce CSF phosphorylated tau in a subject, including CSF p181tau and CSF p217+tau. In some embodiments, the anti-tau antibody is administered in an effective amount to reduce total tau, including total phosphorylated tau (for example, total p181tau, total p217+tau, etc.). In some embodiments, the anti-tau antibody is administered in an effective amount to reduce free tau, including free phosphorylated tau (for example, free p181tau, free p217+tau, etc.). As used herein “free” in the context of tau refers to tau refers to tau that is not bound to an antibody, such as the anti-tau antibody of the present invention.

### EXAMPLE

Embodiments of the present disclosure can be further defined by reference to the following non-limiting examples. It will be apparent to those skilled in the art that many modifications, both to materials and methods, can be practiced without departing from the scope of the present disclosure.

### Example 1

### Safety Pharmacology and Toxicology in Nonclinical Studies

Studies were conducted in rats, minipigs, and monkeys to assess the toxicology and safety of the anti-tau antibody of the present invention.

The anti-tau antibody used in these studies was a humanized IgG1 monoclonal antibody comprising a heavy chain variable region having the amino acid sequence of SEQ ID NO: 25, and a light chain variable region having the amino acid sequence of SEQ ID NO: 26.

### Rats

The toxicity and toxicokinetic profile of the anti-tau antibody was characterized in a study in Sprague Dawley rats (main study: 15/sex/group; toxicokinetics study: 4/sex/group). The animals were administered once weekly IV bolus injections of 0 (PBS), 20, 65, or 200 mg/kg of the anti-tau antibody for two months (nine total doses). Ten rats/sex/group were euthanized on Day 64, with five animals/sex/main study group remaining on study for a six-week recovery period. Animals were evaluated for mortality, clinical signs, body weights, food consumption, ophthalmoscopic findings, clinical pathology parameters (hematology, coagulation, clinical chemistry), gross necropsy, organ weights, and histopathology parameters. In addition, toxicokinetics, anti-drug antibodies (ADAs), and CSF assessment (anti-tau antibody concentrations) were conducted during the study. The results showed that no anti-tau antibody-related effects were observed up to the highest dose of 200 mg/kg, which was considered to be the no-observed-effect level (NOEL). The 200 mg/kg dose was associated with Day 57 mean Cₘₐₓ and AUC_{Day57-64} values of 7,612.21 μg/mL and 17,571.73 μg·day/mL, respectively, in males; and Day 57 mean Cₘₐₓ and AUC_{Day57-64} values of 5,737.42 μg/mL and 10,869.84 μg·day/mL, respectively, in females.

In a separate study, Sprague Dawley rats (main study: 15/sex/group; toxicokinetics study: 5/sex/group) were administered once weekly IV bolus injections of 0 (PBS), 65, 200, or 300 mg/kg of the anti-tau antibody for six months. All surviving animals were euthanized on Day 183, with five animals/sex/main study group remaining on study for a four-week recovery period. Survival, body weight, food consumption, ophthalmic findings, clinical pathology parameters (hematology, coagulation, clinical chemistry), gross necropsy, organ weights and histopathology parameters; toxicokinetics, ADA, and CSF assessment (anti-tau antibody concentrations) were all evaluated in this study. No anti-tau antibody-related effects were seen up to the highest administered dose of 300 mg/kg. Clinical observations were limited to a non-adverse increase in the incidence of red or brown hair discoloration compared to controls. One control male animal was found dead on Day 74 and one 300 mg/kg/week male animal was found dead on Day 170. Although a cause of death was undetermined, the mortality was considered unrelated to the anti-tau antibody because the incidence was comparable between treated and control animals and target organ toxicity was not evident. Administration of the anti-tau antibody by IV bolus injection once weekly for 26 weeks was well tolerated in rats at doses of ≥300 mg/kg. As a result, the 300 mg/kg dose was considered the NOEL and was associated with Day 176 mean Cₘₐₓ and AUC_{Day176-183} exposures of 8416.45 μg/mL and 14723.91 μg·day/mL, respectively (males and females combined).

### Minipigs

The toxicity and toxicokinetics profile of the anti-tau antibody was characterized in a study in Gottingen minipigs® (5/sex/group total). These minipigs were administered once weekly slow bolus IV injections of 0 (PBS), 20, 65, or 200 mg/kg of the anti-tau antibody once a week for six weeks (six doses total), with two animals/sex/group remaining on study for a six-week recovery period. All animals were sedated with Telazol (5 mg/kg IM) prior to dosing. Mortality, clinical signs, body weights, qualitative food consumption, physical, ophthalmoscopic, and electrocardiogram (ECG) examinations, blood pressure, heart rate, respiratory rate, clinical pathology parameters (hematology, coagulation, clinical chemistry, and urinalysis), toxicokinetic parameters, ADA analysis, CSF analysis, gross necropsy, organ weights, and histopathology evaluation were all conducted during the study. No anti-tau antibody-related effects were observed, indicating that the administration of the anti-tau antibody via slow IV bolus injection to male and female minipigs for six weeks was well tolerated at doses ≥200 mg/kg. Based on these results, the NOEL in this study was considered to be 200 mg/kg, which was associated with Day 36 mean combined Cₘₐₓ and AUC_{Day36-43} values in males and females of 3,980.97 μg/mL and 10,017.24 μg·day/mL, respectively.

### Cynomolgus Monkeys

In a non-GLP study, the tolerability and toxicokinetic profile of the anti-tau antibody was characterized in female cynomolgus monkeys (3/group) administered once weekly IV injections of 0 (PBS), 20, 65, or 200 mg/kg of the anti-tau antibody for four weeks (four doses total). Mortality, clinical signs, body weights, qualitative food consumption, veterinary physical examinations, blood pressure, heart rate, respiration rate, clinical pathology parameters (hematology, coagulation, clinical chemistry, urinalysis), toxicokinetic parameters, gross pathology, and organ weights were evaluated during the study. No anti-tau antibody-related changes were observed, indicating that weekly IV doses up to 200 mg/kg were well-tolerated by cynomolgus monkeys. Based on these results, the NOEL in this study was considered to be 200 mg/kg; associated mean Cₘₐₓ and AUC_{Day22-29} values on Day 22 were 4,627.77 μg/mL and 13,303.89 μg·day/mL, respectively.

### Example 2

### Safety of the Anti-Tau Antibody in Humans

A two-part randomized, placebo-controlled, double-blind, single and multiple ascending dose study was performed to investigate safety and tolerability, pharmacokinetics, and pharmacodynamics of an anti-tau antibody of the present invention in healthy subjects and subjects with Alzheimer's Disease. The discussion here will focus on the safety and tolerability results of the study.

The anti-tau antibody used in the study was a humanized IgG1 monoclonal antibody comprising a heavy chain variable region having the amino acid sequence of SEQ ID NO: 25, and a light chain variable region having the amino acid sequence of SEQ ID NO: 26. The anti-tau antibody was supplied as a sterile, preservative-free liquid with a concentration of 50 mg/mL of the antibody in a solution composed of 10 mM histidine, 8.5% (w/v) sucrose, 0.04% (w/v) polysorbate 20, and 20 μg/mL EDTA, at a pH of 5.5.

### Methodology

The study consisted of two parts with nine total cohorts and up to eight subjects in each. Part 1 involved Cohorts 1-5, and Part 2 involved Cohorts A, B, D, and E.

Part 1 was a single ascending dose (SAD) study in healthy subjects. Single ascending IV doses ranging from 1 to 60 mg/kg of the anti-tau antibody or a placebo were administered to sequential cohorts of healthy subjects. Dosing for each cohort in Part 1 occurred over at least two days, with two subjects dosed on the first day (one receiving the placebo, one receiving the anti-tau antibody) and six subjects the following day(s) (one receiving the placebo, five receiving the anti-tau antibody).

Part 2 was a multiple ascending dose (MAD) study in healthy subjects and subjects with prodromal or mild Alzheimer's Disease. Two dose levels (5 mg/kg or 50 mg/kg) of the anti-tau antibody or placebo were evaluated in healthy subjects, and two doses levels (15 mg/kg or 30 mg/kg) of the anti-tau antibody or placebo were evaluated in subjects with prodromal or mild Alzheimer's Disease, as multiple ascending IV doses over a period of eight weeks (IV dosing occurred on Day 1, Day 29, and Day 57). If two or more subjects were available for dosing at the initiation of any given MAD cohort in Part 2, then sentinel dosing was done (as described for Part 1), with one subject receiving placebo and one subject receiving the prior to additional subjects being dosed.

The subjects in Part 1 were male and female, 55 to 75 years of age, inclusive, and healthy. The subjects in Part 2 were male and female, 55 to 80 years of age inclusive, and included healthy subjects and subjects with prodromal or mild Alzheimer's Disease. Alzheimer's Disease subjects had a CDR-GS of 0.5 or 1.0 consistent with mild cognitive impairment (MCI; prodromal Alzheimer's Disease) or mild Alzheimer's Disease, respectively, as well as evidence of amyloid deposition and tauopathy as demonstrated by an abnormal CSF Aβ1-42 and elevated CSF p181tau.

For Part 1, dosages of 1 mg/kg, 3 mg/kg, 10 mg/kg, 30 mg/kg, and 60 mg/kg were administered for the various treatment arms. For Part 2, dosages of 5 mg/kg, 15 mg/kg, 30 mg/kg, and 50 mg/kg were administered for the various treatment arms. For both parts, the placebo was supplied as a 0.9% sodium chloride solution.

Following dosing and after completion of the inpatient phase, subjects from Part 1 returned to the study site for regular follow-up visits up to 13 weeks following dosing to assess safety and tolerability, as well as efficacy (not discussed here). Subjects from Part 2 returned for subsequent dose administrations on Day 29 and Day 57 and for regular follow-up visits up to 13 weeks following last dosing to assess safety and tolerability, as well as efficacy (not discussed here)

Sampling schemes varied by cohort and were balanced across treatment groups to characterize the pharmacokinetic profile of the anti-tau antibody and assess the biomarker response.

Completion of the Day 92 (Week 13) visit for Part 1 and Day 148 (Week 21) visit for Part 2 constituted the end of participation in the study unless a CSF sample was collected at that visit. In that case, the subject had an additional safety follow-up visit at Day 106 (Week 15) for Part 1 or Day 162 (Week 23) for Part 2.

Safety and tolerability assessments included vital signs, safety labs, magnetic resonance imaging (MRI) of the brain, 12-lead ECG, and telemetry (Part 1 only).

Safety and Tolerability Results

There were no deaths reported during the study and no early terminations due to treatment-emergent adverse events (TEAEs). Serious adverse events were reported in two subjects: in Part 1, a healthy subject treated with placebo experienced post lumbar puncture syndrome/suspected post spinal headache and hypertension; and in Part 2, an Alzheimer's Disease subject treated with the 15 mg/kg anti-tau antibody dose experienced renal neoplasm, although this adverse event was not considered related to the treatment with the anti-tau antibody.

All subjects who received at least one dose of study intervention were included in the safety analysis set. In Part 1 of the study, 24 (80%) of the 30 subjects treated with the anti-tau antibody reported one or more adverse events (AEs); 50% of subjects treated with 1 mg/kg, 66.7% of subjects treated with 3 mg/kg, 100% of subjects treated with 10 mg/kg, 83.3% of subjects treated with 30 mg/kg, and 100% of subjects treated with 60 mg/kg. Of the ten subjects treated with placebo, eight (80%) reported one or more AEs.

In Part 1 of the study, the most commonly reported TEAEs (>20% of subjects) were post lumbar puncture syndrome in subjects who received the 1 mg/kg dose of the anti-tau antibody; post lumbar puncture syndrome, hypercholesterolemia, headache, nausea, and hot flush in subjects who received the 10 mg/kg dose of the anti-tau antibody; hepatic enzyme increase in subjects who received the 30 mg/kg dose of the anti-tau antibody; headache, hypercholesterolemia, post lumbar puncture syndrome, procedural pain, muscle spasms, and neck pain in subjects who received the 60 mg/kg dose of the anti-tau antibody; and headache and back pain in subjects who received placebo. No TEAEs were reported in more than one subject who received the 3 mg/kg dose of the anti-tau antibody.

In Part 2 of the study, 20 (87%) of the 23 subjects treated with the anti-tau antibody reported one or more AEs; 66.7% of subjects treated with 5 mg/kg, 83.3% of subjects treated with 15 mg/kg, 100% of subjects treated with 30 mg/kg, and 100% of subjects treated with 50 mg/kg. Of the six subjects treated with placebo, five (83.3%) reported one or more AEs.

In Part 2 of the study, the most commonly reported TEAEs (>20% of subjects) were back pain and headache in subjects who received the 15 mg/kg dose of the anti-tau antibody; headache and post lumbar puncture syndrome in subjects who received the 50 mg/kg dose of the anti-tau antibody; and headache and fatigue in subjects who received placebo. No TEAEs were reported in more than one subject who received the 5 mg/kg dose or the 30 mg/kg dose of the anti-tau antibody.

No clinically important abnormalities were observed in any of the laboratory values, vital sign parameters, or brain MRIs.

Thus, these results show that, overall, the anti-tau antibody was generally safe and well tolerated in healthy adults and in subjects with prodromal or mild Alzheimer's Disease.

### Example 3

### Efficacy and Safety of the Anti-Tau Antibody in Humans with Early Alzheimer's Disease.

A randomized, placebo-controlled, double-blind, parallel-group study is performed to assess the efficacy and safety of an anti-tau antibody of the present invention in subjects with early Alzheimer's Disease.

### Objectives

Primary objective: to evaluate the effect of the anti-tau antibody versus placebo on cognitive decline as measured by the integrated Alzheimer's Disease Rating Scale (iADRS), a composite of cognition and function.

Key secondary objectives relating to cognition and function: to evaluate the effect of the anti-tau antibody versus placebo on cognitive decline as measured by the Alzheimer's Disease Assessment Scale Cognitive, subscale 13-item version (ADAS-Cog13); and to evaluate changes in functional status between subjects treated with the anti-tau antibody or placebo as measured by the Alzheimer's Disease Cooperative Study Activities of Daily Living for Mild Cognitive Impairment (ADCS-ADL-MCI).

Secondary objectives relating to cognition and function: to evaluate the effect of the anti-tau antibody compared with placebo on cognitive decline, as measured by the Repeatable Battery for the Assessment of Neuropsychological Status (RBANS) Total Scale Index Score; to evaluate the effect of the anti-tau antibody compared with placebo as measured by the 5 RBANS indices and 12 subtests comprising the RBANS; to evaluate if treatment with the anti-tau antibody slows clinical progression compared with placebo as measured by the CDR scale—sum of boxes (CDR-SB); to evaluate changes in neuropsychiatric/behavioral status between subjects treated with the anti-tau antibody or placebo as measured by the Neuropsychiatric Inventory (NPI); and to evaluate the effect of the anti-tau antibody compared with placebo on proportion of subjects progressing from CDR-GS 0 to 0.5 or higher, 0.5 to 1 or higher, or 1 to 2 or higher, from baseline to post-baseline.

Secondary objectives relating to biomarkers, pharmacokinetics, and immunogenicity: to evaluate the effect of the anti-tau antibody on the accumulation and/or propagation of tau pathology compared with placebo, as measured by tau PET; to evaluate the effect of the anti-tau antibody on levels of total, free, and bound p217+tau fragments in CSF; to evaluate the peripheral and central exposure (pharmacokinetics) of the anti-tau antibody following chronic treatment; and to evaluate the immunogenicity (presence of anti-drug antibodies (ADAs) in serum) of the anti-tau antibody following chronic treatment.

Secondary objectives relating to safety outcomes: to investigate the safety and tolerability of the anti-tau antibody in subjects with Early Alzheimer's Disease, as assessed by AEs, SAES, early discontinuations due to AEs, ECGs, laboratory evaluations, physical and neurological examinations, vital signs, and Columbia Suicide Severity Rating Scale (C-SSRS), and brain MRI is included for safety evaluation.

Exploratory objectives: to evaluate changes in functional status between subjects treated with the anti-tau antibody versus placebo as measured by the Amsterdam Instrumental Activities of Daily Living Questionnaire (IADL); to evaluate changes in quality of life between subjects treated with the anti-tau antibody versus placebo as measured by the Quality of Life in Alzheimer's Disease (QOL-AD); to evaluate the relationship between dose and pharmacokinetics of the anti-tau antibody on clinical efficacy, safety, and biomarker assessments; to evaluate the relationship between tau PET burden and CSF and plasma phosphorylated tau (p181tau and p217+tau) levels; to evaluate the relationship between CSF and plasma amyloid levels; to evaluate the effect of the anti-tau antibody on changes in brain volume as measured by volumetric MRI; to explore the effects of the anti-tau antibody on markers of Aβ pathophysiology (e.g., Aβ42, Aβ40, and Aβ42/Aβ40) and downstream markers of neuronal injury, neurodegeneration (e.g., neurofilament light chain (NfL), neurogranin), and inflammation (e.g., chitinase-3-like protein 1 (YKL40), soluble triggering receptor expressed on myeloid cells 2 (TREM2), etc.) in CSF and/or plasma/serum compared with placebo; to explore the potential relationship of biomarkers of tau and neurodegeneration (CSF phosphorylated tau, total tau, NfL, neurogranin, tau PET, volumetric MRI) with change in clinical decline; and to evaluate differences in resource utilization (caregiver time, hospitalizations, changes in housing, etc.) between subjects treated with the anti-tau antibody or placebo as measured by Resource Utilization in Dementia Lite (RUD-Lite).

### Study Design

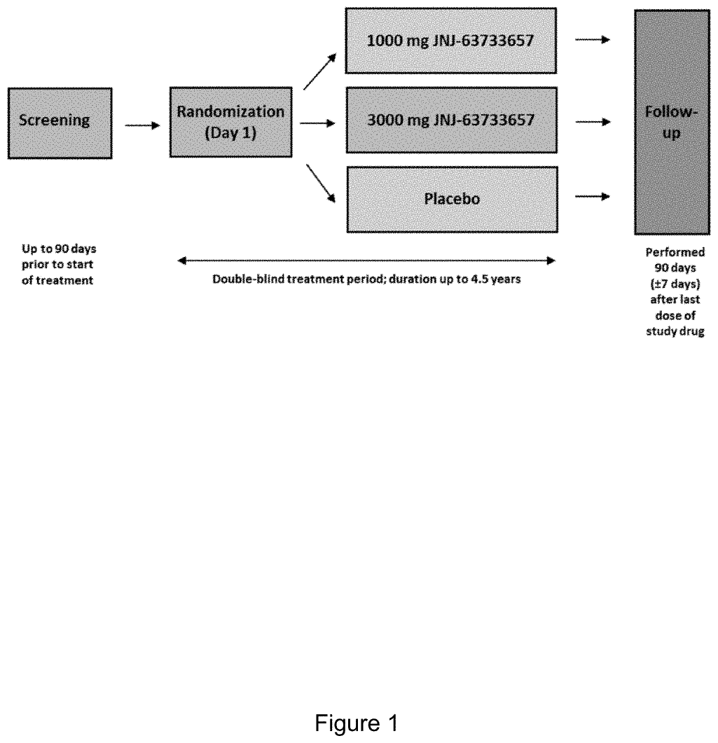

A schematic overview of the study is provided in FIG. 1. For all enrolled subjects, the study consists of:
(a) a 13-week (90-day) screening period (can be extended up to 120 days with prior approval from medical monitor);
(b) a variable double-blind treatment period of up to 232 weeks (4.5 years); and
(c) a follow-up period of approximately 13 weeks (90 days).

This study is an outpatient study. The double-blind treatment period is of variable duration, continuing until all subjects have had the opportunity to receive double-blind treatment for up to 128 weeks. Study subjects are followed in the double blind period for a maximum duration of up to 232 weeks (4.5 years), with longest follow-up for those subjects enrolled earliest.

### Study Population

Screening for eligible subjects is performed within 90 days before the administration of the study intervention (i.e., the anti-tau antibody or placebo).

The study is enrolling approximately 420 subjects, approximately 140 subjects per treatment group. The target population consists of subjects aged 55 to 80 years, inclusive at the time of initial consent, with sporadic Early Alzheimer's Disease, with biomarker evidence of pathological phosphorylated tau protein (evaluated first by plasma prescreen and confirmed by pathologic tau on tau PET) (T+).

The inclusion criteria is as follows:
(1) 55 to 80 years of age, inclusive, at the time of initial consent.
(2) Early Alzheimer's Disease: gradual and progressive subjective decline in the subject's cognition over at least the past 6 months, as reported by the subject and informant (study partner) and CDR-GS of 0.5 and memory box score ≥0.5 at screening.
(3) Evidence of pathologically elevated tau (T+) as defined first in plasma. Only plasma T+ subjects will undergo a tau PET scan at screening to confirm T+ status.
(4) Evidence of pathologic tau on a screening tau PET scan reviewed centrally by a qualified reader.
(5) Able to read and write and with a minimum 5 years of formal education as reported by subject and study partner at screening.
(6) Willing to participate in this study (signed written informed consent) and to comply with the study protocol.
(7) Have a designated study partner who has adequate literacy to participate and be judged to have high likelihood of completing the study with the subject.
(8) Female subjects must not be of childbearing potential; that is, they must be either:
   (a) postmenopausal (no menses for 1 year without an alternative medical cause; high follicle stimulating hormone (FSH) level (>40 IU/L or mIU/mL) in the postmenopausal range may be used to confirm a postmenopausal state in women not using hormonal contraception or hormonal replacement therapy, however, in the absence of 1 year of amenorrhea, a single FSH measurement is insufficient); or
   (b) permanently sterilized (e.g., tubal occlusion, hysterectomy, bilateral salpingectomy); or
   (c) otherwise be incapable of pregnancy.
(9) Male subjects who are sexually active with a woman of childbearing potential must agree to use a barrier method of contraception (e.g., condom with spermicidal foam/gel/film/cream/suppository or partner with occlusive cap (diaphragm or cervical/vault caps) with spermicidal foam/gel/film/cream/suppository) during the study and up to 16 weeks after the last dose of study intervention; in addition, their female partner should also use a highly effective method of birth control (e.g., hormonal contraception) for at least the same duration; a male study subject whose female partner is pregnant should use a condom during the study and up to 16 weeks after the last dose of study intervention.
10. Male subjects must agree not to donate sperm during the study and up to 16 weeks after the last dose of study intervention.

The exclusion criteria is as follows:
1. Subjects with CDR-GS ≥2 at predose baseline CDR administration.
2. Subjects who fulfill diagnostic criteria for MCI or dementia/mild or major neurocognitive disorder suspected to be due to any etiology other than Alzheimer's Disease (e.g., MCI/dementia due to frontotemporal lobar degeneration, diffuse Lewy body disease, Parkinson's disease, cerebrovascular disease, normal pressure hydrocephalus, head injury, drug or alcohol abuse/dependence, anoxic brain injury, etc.).
3. Geriatric Depression Scale (GDS) 30 score ≥12.
4. Hachinski Ischemic Scale (HIS) >4.
5. Known carriers of a Presenilin-1 (PSEN1), PSEN2, or Amyloid Precursor Protein mutation associated with Autosomal Dominant Alzheimer's Disease or any other neurodegenerative disease.
6. Subjects with extensive, widespread tau pathology, as measured by tau PET.
7. Has received acetylcholinesterase inhibitors, memantine, and/or other permitted Alzheimer's Disease therapy for less than four months or has less than two months of a stable dose on these treatments by the start of screening. (Note: if a subject has recently stopped acetylcholinesterase inhibitors, and/or memantine, he or she must have discontinued treatment at least two months before the start of screening). Concomitant use of Alzheimer's Disease therapies that target the underlying pathophysiology of Alzheimer's Disease (e.g., anti-amyloid or anti-tau therapies) are not permitted.
8. Has received medications that affect the central nervous syndrome (CNS), except treatments for Alzheimer's Disease (as detailed in exclusion criterion (7)), for less than two months; that is, doses of chronic medications that effect the CNS should be stable for at least two months before the start of screening. Chronic use of benzodiazepines is not permitted.
9. Presence of any neurological, psychiatric, or medical conditions associated with a long-term risk of significant cognitive impairment or dementia including, but not limited to, pre-manifest Huntington's disease, multiple sclerosis, Parkinson's disease, Down's syndrome, active alcohol/drug abuse or major psychiatric disorders including, but not limited to, schizophrenia, schizoaffective disorder, or bipolar affective disorder or current episode of major depressive disorder.
10. Presence of thyroid disease or dysfunction, defined as a thyroid-stimulating hormone (TSH) value that is outside central laboratory's normal range for TSH (i.e., below the lower limit of normal or higher than the upper limit of normal); or vitamin B12 or folic acid deficiency, defined as a vitamin B12 or folate value that is below the central laboratory's lower limit of normal. Subjects may be rescreened if treated and have evidence of thyroid stimulating hormone, vitamin B12, and folic acid levels within normal range for at least three months.
11. History of epilepsy, fits, or unexplained blackouts other than vasovagal syncope within ten years before screening.
12. Known allergies, hypersensitivity, or intolerance to the anti-tau antibody or formulation elements.
13. History of substance use disorder according to most current version of the Diagnostic and Statistical Manual of Mental Disorders criteria within the past five years before screening or positive test result(s) for other drugs of abuse (including barbiturates, opiates, cocaine, amphetamines, and benzodiazepines) at screening (except if related to current treatment).
14. Any current medical conditions that, in the opinion of the investigator, are clinically significant and might make the subject's participation in an investigational study unsafe, e.g., uncontrolled or unstable disease of any major organ system; history within the last six months of any acute illness of a major organ system requiring emergency care or hospitalization, including revascularization procedures; severe renal or hepatic failure; unstable or poorly controlled diabetes mellitus, hypertension, or heart failure; malignant neoplasms within the last three years (except for basal or squamous cell carcinoma in situ of the skin, or cervix in female subjects, or localized prostate cancer in male subjects that, in the opinion of the investigator, is considered cured with minimal risk of recurrence); any clinically relevant abnormalities in blood parameters included in local site routine assessments; severe loss of vision, hearing or communicative ability.
15. Any conditions or planned prolonged periods of absence (e.g., vacation) preventing cooperation or completion of the required assessments in the study, as judged by the investigator.
16. Clinically significant abnormal physical or neurological examination, vital signs at screening or baseline (Day 1 predose), or laboratory findings at screening. Subjects may be rescreened if they meet inclusion criteria and do not meet any exclusion criteria after findings are treated and the subject is medically stable for at least three months.
17. Has alanine aminotransferase (ALT) ≥2×upper limit of normal (ULN), aspartate aminotransferase (AST) ≥3×ULN, and/or total bilirubin ≥2×ULN at screening. Subjects with diagnosed Gilbert's Syndrome are permitted.
18. History of a positive test for human immunodeficiency virus (HIV) antibody, or tests positive for HIV at screening.
19. QT interval corrected for heart rate using Bazett's formula (QTcB)>450 msec (males) or >470 msec (females), as evaluated by the central ECG vendor at screening and by the Principal Investigator at Day 1, predose. ECGs will be performed in triplicate and subjects will be excluded if more than 1 of the 3 QTcB measurements are >450 msec (males) or >470 msec (females). Note: ECG measurements may be repeated once; for any potentially clinically significant findings, the site will manage the subject as per standard clinical practice.
20. Any contraindications for MRI.
21. Any evidence of intracranial pathology which, in the opinion of the investigator or the sponsor (as outlined in the MRI charter), may affect cognition including, but not limited to, brain tumors (benign or malignant), aneurysm or arteriovenous malformations, territorial stroke (excluding smaller watershed strokes), recent hemorrhage (parenchymal or subdural), or obstructive hydrocephalus. Subjects with an MRI scan demonstrating markers of small vessel disease (e.g., white matter changes or lacunar infarcts) judged to be clinically insignificant, or microbleeds are allowed.
22. Signs of increased intracranial pressure (e.g., based on clinical or MRI examination).
23. As determined by the Principal Investigator, subject is participating in another clinical trial or other medical research that is not scientifically or medically compatible with this study at screening or for the duration of their participation in the current study.
24. Subject has received an investigational drug (including passive immunization) or used an investigational medical device for Alzheimer's Disease within three months or five half-lives, whichever is longest, before the baseline visit (Day 1), or has previously completed or withdrawn from this study or other anti-tau antibody studies.
25. Subject has previously received an active vaccine directed to tau.
26. Diminished decision-making capacity that renders the individual not capable of consenting or completing study assessments in the opinion of the Principal Investigator.
27. History of any suicidal behavior (attempt, interrupted, aborted, or preparatory) in the past six months prior to screening.
28. Past or planned exposure to ionizing radiation that in combination with the planned administration with study tau PET ligand would result in a cumulative exposure that exceeds local recommended exposure limits.
29. Is an employee of the investigator or study site with direct involvement in the proposed study or other studies under the direction of that investigator or study site, or is a family member of an employee or the investigator.
30. Currently resides in a residential nursing facility. Subjects who must be admitted for rehabilitation to a nursing facility during the study may continue in the study if they are able to complete study procedures.
31. Does not have good venous access, precluding frequent blood draws and IV infusions every four weeks.
32. Any other factors in the opinion of the investigator and/or sponsor that could contraindicate participation in the study or suggest inappropriate clinical rage of Alzheimer's Disease (e.g., discordance of CDR-GS and RBANS Delayed Memory Index (DMI)).
33. Planning to take, or currently taking, an approved treatment that targets the underlying pathophysiology of Alzheimer's Disease (e.g., anti-amyloid therapies). If a participant has discontinued an approved treatment that targets the underlying pathophysiology of Alzheimer's Disease (e.g., anti-amyloid therapies), there must be at least 3 months or 5 half-lives, whichever is longest, between the last dose of the treatment and Day 1 of the double-blind treatment period.

### Treatment Period

Subjects are assigned randomly (central randomization) to one of the following three treatment groups in a 1:1:1 ratio:
(i) 1000 mg dosage of the anti-tau antibody;
(ii) 3000 mg dosage of the anti-tau antibody; or
(iii) placebo.

The anti-tau antibody is a humanized IgG1 monoclonal antibody comprising a heavy chain variable region having the amino acid sequence of SEQ ID NO: 25, and a light chain variable region having the amino acid sequence of SEQ ID NO: 26. The anti-tau antibody is supplied as a sterile, preservative-free liquid with a concentration of 50 mg/mL of the antibody in a solution composed of 10 mM histidine, 8.5% (w/v) sucrose, 0.04% (w/v) polysorbate 20, and 20 μg/mL EDTA, at a pH of 5.5.

The formulation for the placebo is similar to the anti-tau antibody formulation, but without the antibody.

The anti-tau antibody or placebo is administered intravenously every 4 weeks. Infusions take place at a constant rate over 60 minutes. Subjects continue treatment with the assigned study intervention until all randomized subjects have had the opportunity to receive up to 128 weeks of double-blind treatment, at which time the study intervention will be discontinued for all subjects. The maximum duration of double-blind period treatment for any subject will be 232 weeks (4.5 years).

### Study Assessments

The following assessments are included during the study:
iADRS: change from baseline on the iADRS is the primary endpoint to evaluate the effect of the anti-tau antibody versus placebo on clinical decline.
ADAS-Cog13: change from baseline on the ADAS-Cog13 is a key secondary endpoint to evaluate the effect of the anti-tau antibody versus placebo on cognitive decline.
ADCS-ADL-MCI: change from baseline on the ADCS-ADL-MCI is a key secondary endpoint to evaluate changes in functional status between subjects treated with the anti-tau antibody or placebo.
RBANS Total Scale Index Score: change from baseline on the RBANS Total Scale Index Score is a secondary endpoint to evaluate the effect of the anti-tau antibody compared with placebo on cognitive decline.
5 individual RBANS indices and 12 subtests comprising the RBANS: change from baseline in each of the 5 individual RBANS indices and each of the 12 subtests comprising the RBANS is a secondary endpoint to evaluate the effect of the anti-tau antibody compared with placebo.
CDR-SB: change from baseline on the CDR-SB is a secondary endpoint to evaluate if treatment with the anti-tau antibody slows clinical progression compared with placebo.
NPI: change from baseline in NPI is a secondary endpoint to evaluate changes in neuropsychiatric/behavioral status between subjects treated with the anti-tau antibody or placebo.
CDR-GS: proportion of subjects progressing from CDR-GS 0 to 0.5 or higher, 0.5 to 1 or higher, or 1 to 2 or higher, from baseline to post-baseline, is a secondary endpoint to evaluate the effect of the anti-tau antibody compared with placebo.
Tau PET: change from baseline in brain tau burden, as measured by tau PET, is a secondary endpoint to evaluate the effect of the anti-tau antibody on the accumulation and/or propagation of tau pathology compared with placebo.
CSF concentrations of total, free, and bound p217+tau fragments: change from baseline in CSF concentrations of total, free, and bound p217+tau fragments is a secondary endpoint to evaluate the effect of the anti-tau antibody on levels of total, free, and bound p217+tau fragments in CSF.
CSF and serum concentrations of the anti-tau antibody: CSF and serum concentrations of the anti-tau antibody at different time points (weeks 52, 104, 208 for CSF concentrations; weeks 4, 8, 12, 16, 20, 24, 36, 52, 76, 104, 128, 156, 180, 208, and 232 for serum concentrations) is a secondary endpoint to evaluate the peripheral and central exposure (PK) of the anti-tau antibody following chronic treatment.
ADA in serum: ADA in serum at different time points (up to 245 weeks (90 days, ±7 days, after last dose of study intervention) is a secondary endpoint to evaluate the immunogenicity of the anti-tau antibody following chronic treatment.
AEs, SAEs, ECGs, laboratory evaluations, physical and neurological examination, vital signs, brain MRI, and C-SSRS: nature, frequency, severity, and timing of AEs, discontinuations due to AEs, and SAEs, and evaluation of other safety parameters as measured by 12-lead ECGs (performed in triplicate), laboratory evaluations (including hematology, chemistry, and urinalysis), complete physical and neurological examination, vital signs (including supine and standing systolic and diastolic blood pressure and pulse, temperature, and weight), brain MRI, assessment of suicidality with C-SSRS, are a secondary endpoint to investigate the safety and tolerability of the anti-tau antibody in subjects with early AD.
Amsterdam IADL: change from baseline on the Amsterdam IADL is an exploratory endpoint to evaluate changes in functional status between subjects treated with the anti-tau antibody versus placebo.
QOL-AD: change from baseline on the QOL-AD is an exploratory endpoint to evaluate changes in quality of life between subjects treated with the anti-tau antibody versus placebo.
Anti-tau antibody dose and serum and CSF levels, and efficacy, safety, and biomarker findings: correlation of the anti-tau antibody dose and serum and CSF levels with efficacy, safety and biomarker findings of note are an exploratory endpoint to evaluate the relationship between dose and PK of the anti-tau antibody on clinical efficacy, safety, and biomarker assessments.
CSF and plasma concentrations of p181tau and p217+tau and tau PET: correlation/concordance of baseline and change from baseline in CSF and plasma concentrations of p181tau and p217+tau and tau PET is an exploratory endpoint to evaluate the relationship between tau PET burden and CSF and plasma phosphorylated tau (p181tau and p217+tau) levels.
CSF Aβ and plasma Aβ: correlation/concordance between CSF Aβ and plasma Aβ at baseline and change from baseline is an exploratory endpoint to evaluate the relationship between CSF and plasma amyloid level.
Volumetric MRI: change from baseline in hippocampal, whole brain, and ventricular volume using MRI is an exploratory endpoint to evaluate the effect of the anti-tau antibody on changes in brain volume.
Aβ pathophysiology, neuronal injury, and neurodegeneration, and biomarkers of inflammation measured in CSF and/or plasma/serum: change from baseline in Aβ pathophysiology (Aβ₄₂, Aβ₄₀, and Aβ₄₂/Aβ₄₀), neuronal injury, and neurodegeneration (NfL, neurogranin) or biomarkers of inflammation (YKL40, soluble TREM2) as measured in CSF and/or plasma/serum is an exploratory endpoint explore the effects of the anti-tau antibody on markers of Aβ pathophysiology and downstream markers of neuronal injury, neurodegeneration, and inflammation in CSF and/or plasma/serum compared with placebo.
CSF p-tau, t tau, NfL, neurogranin, tau PET, volumetric MRI, CDR SB, iADRS, RBANS, and/or ADAS Cog13: correlation between baseline and change from baseline in CSF p-tau, t tau, NfL, neurogranin, tau PET, volumetric MRI and change from baseline in clinical decline (CDR SB and iADRS) or cognitive score (RBANS and ADAS Cog13) is an exploratory endpoint to explore the potential relationship of biomarkers of tau and neurodegeneration (CSF p-tau, t-tau, NfL, neurogranin, tau PET, volumetric MRI) with change in clinical decline.
Resource utilization: change from baseline in resource utilization (e.g., caregiver time, hospitalizations, changes in housing) on RUD Lite is an exploratory endpoint to evaluate differences in resource utilization (caregiver time, hospitalizations, changes in housing, etc) between subjects treated with the anti-tau antibody or placebo.

### Post-Treatment Period

Approximately 90 days (±7 days) after the last dose of study intervention in the double-blind treatment period (i.e., after the last visit of the double-blind treatment period), subjects return to the site for a follow-up visit, if not entering an open-label extension study. The procedures completed during the follow-up visit include a physical examination, neurological examination, assessment of vital signs, hematology, chemistry, and urinalysis. Subjects who withdraw prematurely from the study during the double-blind treatment period are also expected to complete the post-treatment period (follow-up visit) assessments within approximately 90 days (±7 days) after the last dose of study intervention, or the early termination visit assessments, whichever comes last.

If the subject remains in the double-blind treatment period (without study medication) for more than 90 days after the last dose of study medication, a safety follow-up visit is not required after completing the double-blind treatment period. If the subject remains in the double-blind treatment period (without study medication) for a period of time but discontinues this phase prior to having reached 90 days after the last dose of study medication, an Early Termination visit should be performed, followed by a safety follow-up visit approximately 90 days after the last dose of study medication.

At the last visit, the detailed reasons for study and study intervention discontinuation are collected.

Investigators may recontact the subject or study partner to obtain long-term follow-up information to determine safety or survival status. If the subject has died, the date and cause of death are collected and documented.

### REFERENCES

Abhinandan K R and Martin A C R. Analysis and improvements to Kabat and structurally correct numbering of antibody variable domains. Mol. Immunol. 45: 3832-3839 (2008).
Almagro J C. Identification of differences in the specificity-determining residues of antibodies that recognize antigens of different size: implications for the rational design of antibody repertoires. J. Mol. Recognit. 17: 132-143 (2004).
Alonso A, et al. Hyperphosphorylation induces self-assembly of tau into tangles of paired helical filaments/straight filaments. Proc. Natl. Acad. Sci. USA. 98: 6923-6928 (2001).
Asuni A A, et al. Immunotherapy targeting pathological tau conformers in a tangle mouse model reduces brain pathology with associated functional improvements. J. Neurosci. 27: 9115-9129 (2007).
Bierer L M, et al. Neocortical neurofibrillary tangles correlate with dementia severity in Alzheimer's disease. Arch. Neurol. 52: 81-88 (1995).
Boutajangout A, et al. Passive immunization targeting pathological phospho-tau protein in a mouse model reduces functional decline and clears tau aggregates from the brain. J. Neurochem. 118: 658-667 (2011).
Braak H and Braak E. Neuropathological stageing of Alzheimer-related changes. Acta Neuropathol. 82: 239-259 (1991).
Berg L. Clinical Dementia Rating (CDR). Psychopharmacol. Bull. 24: 637-639 (1988).
Brunden K R, et al. Advances in tau-focused drug discovery for Alzheimer's disease and related tauopathies. Nat. Rev. Drug Discov. 8: 783-793 (2009).
Butner K A and Kirschner M W. Tau protein binds to microtubules through a flexible array of distributed weak sites. J. Cell. Biol. 115: 717-730 (1991).
Chothia C. and Lesk M. Canonical structures for the hypervariable regions of immunoglobulins. J. Mol. Biol. 196: 901-917 (1987).
Delacourte A. The molecular parameters of tau pathology. Tau as a killer and a witness. Adv. Exp. Med. Biol. 487: 5-19 (2001).
Fishwild D M, et al. High-avidity human IgG kappa monoclonal antibodies from a novel strain of minilocus transgenic mice. Nat. Biotechnol. 14: 845-51 (1996).
Friedhoff P, et al. Structure of tau protein and assembly into paired helical filaments. Biochim. Biophys. Acta. 1502: 122-132 (2000).
Goedert M, et al. Neurofibrillary tangles and beta-amyloid deposits in Alzheimer's disease. Curr. Opin. Neurobiol. 1: 441-447 (1991).
Hanger D P, et al. Tau phosphorylation: the therapeutic challenge for neurodegenerative disease. Trends Mol. Med. 15: 112-119 (2009).

Knappik A., et al. Fully synthetic human combinatorial antibody libraries (HuCAL) based on modular consensus frameworks and CDRs randomized with trinucleotides. J. Mol. Biol. 296: 57-86 (2000).
Krebs B, et al. High-throughput generation and engineering of recombinant human antibodies. J. Immunol. Methods. 254: 67-84 (2001).
Lefranc M P, et al. IMGT unique numbering for immunoglobulin and T cell receptor variable domains and Ig superfamily V-like domains. Dev. Comp. Immunol. 27: 55-77 (2003).
Lonberg N, et al. Antigen-specific human antibodies from mice comprising four distinct genetic modifications. Nature. 368: 856-859 (1994).
Martin A C R. Antibody Engineering. Kontermann R and Dubel S eds., Springer-Verlag, Berlin, 2:33-51 (2010).
Mendez M J, et al. Functional transplant of megabase human immunoglobulin loci recapitulates human antibody response in mice. Nat. Genet. 15: 146-56 (1997).
Remington's Pharmaceutical Science. Osol A and Hoover J E eds., Mack Publishing Company, Easton, Pa. (15th ed. 1980).
Schroeder S K, et al. Tau-directed immunotherapy: a promising strategy for treating Alzheimer's disease and other tauopathies. J. Neuroimmune Pharmacol. 11: 9-25 (2016).
Shi L, et al. De novo selection of high-affinity antibodies from synthetic fab libraries displayed on phage as pIX fusion proteins. J. Mol. Biol. 397: 385-396 (2010).
Sigurdsson E M. Tau immunotherapy. Neurodegener. Dis. 16: 34-38 (2016).
Wu T T and Kabat E. An analysis of the sequences of the variable regions of Bence Jones proteins and myeloma light chains and their implications for antibody complementarity. J. Exp. Med. 132: 211-250 (1970).

1. A method of administering a monoclonal antibody to a subject in need thereof, the method comprising administering to the subject a pharmaceutical composition comprising the monoclonal antibody and a pharmaceutically acceptable carrier,
wherein the monoclonal antibody is administered in an amount of about 500 mg to 5000 mg per dose, and
wherein the monoclonal antibody comprises a heavy chain variable complementarity-determining region (CDR) 1 comprising the amino acid sequence of SEQ ID NO: 1, a heavy chain variable CDR2 comprising the amino acid sequence of SEQ ID NO: 2, a heavy chain variable CDR3 comprising the amino acid sequence of SEQ ID NO: 3, a light chain variable CDR1 comprising the amino acid sequence of SEQ ID NO: 13, a light chain variable CDR2 comprising the amino acid sequence of SEQ ID NO: 14, and a light chain variable CDR3 comprising the amino acid sequence of SEQ ID NO: 15.

2. (canceled)

3. The method of claim 1, wherein the monoclonal antibody comprises a heavy chain variable CDR1 having the amino acid sequence of SEQ ID NO: 1, a heavy chain variable CDR2 having the amino acid sequence of SEQ ID NO: 2, a heavy chain variable CDR3 having the amino acid sequence of SEQ ID NO: 3, a light chain variable CDR1 having the amino acid sequence of SEQ ID NO: 13, a light chain variable CDR2 having the amino acid sequence of SEQ ID NO: 14, and a light chain variable CDR3 having the amino acid sequence of SEQ ID NO: 15.

4. The method of claim 1, wherein the monoclonal antibody comprises a heavy chain variable region comprising the amino acid sequence of SEQ ID NO: 25, and a light chain variable region comprising the amino acid sequence of SEQ ID NO: 26.

5. The method of claim 4, wherein the monoclonal antibody comprises a heavy chain variable region having the amino acid sequence of SEQ ID NO: 25, and a light chain variable region having the amino acid sequence of SEQ ID NO: 26.

6. The method of claim 1, wherein the monoclonal antibody comprises a heavy chain comprising the amino acid sequence of SEQ ID NO: 27, and a light chain comprising the amino acid sequence of SEQ ID NO: 28.

7. The method of claim 6, wherein the monoclonal antibody comprises a heavy chain having the amino acid sequence of SEQ ID NO: 27, and a light chain having the amino acid sequence of SEQ ID NO: 28.

8. The method of claim 1, wherein the pharmaceutical composition further comprises histidine, sucrose, polysorbate 20, and ethylenediamine tetra-acetic acid.

9. The method of claim 1, wherein the pharmaceutical composition has a pH of about 5-6.

10. The method of claim 1, wherein the monoclonal antibody is administered in an amount of about 1000 mg to about 3000 mg per dose.

11. The method of claim 1, wherein the monoclonal antibody is administered in an amount of about 2000 mg to about 5000 mg per dose.

12. The method of claim 11, wherein the monoclonal antibody is administered in an amount of about 3000 mg to about 5000 mg per dose.

13. The method of claim 1, wherein the monoclonal antibody is administered in an amount of about 500 mg, 750 mg, 1000 mg, 1200 mg, 1250 mg, 1400 mg, 1500 mg, 1600 mg, 1750 mg, 1800 mg, 2000 mg, 2200 mg, 2250 mg, 2400 mg, 2500 mg, 2600 mg, 2750 mg, 2800 mg, 3000 mg, 3200 mg, 3250 mg, 3400 mg, 3500 mg, 3600 mg, 3750 mg, 3800 mg, 4000 mg, 4200 mg, 4250 mg, 4400 mg, 4500 mg, 4600 mg, 4750 mg, 4800 mg, or 5000 mg, or any value in between, per dose.

14. The method of claim 13, wherein the monoclonal antibody is administered in an amount of about 1000 mg per dose.

15. The method of claim 13, wherein the monoclonal antibody is administered in an amount of about 3000 mg per dose.

16. The method of claim 13, wherein the monoclonal antibody is administered in an amount of about 4000 mg per dose.

17. The method of claim 13, wherein the monoclonal antibody is administered in an amount of about 5000 mg per dose.

18. The method of claim 1, wherein the pharmaceutical composition is administered by intravenous infusion.

19. The method of claim 1, wherein the pharmaceutical composition is administered as more than one dose.

20. The method or pharmaceutical composition of claim 19, wherein the administration of each dose is separated by a period of about 4 weeks.

21. The method of claim 1, wherein the subject in need of a treatment of Alzheimer's Disease.

22. The method of claim 1, wherein the subject is in need of a treatment of early Alzheimer's Disease, prodromal Alzheimer's Disease, or mild Alzheimer's Disease.
